(19) United States
(12) Patent Application Publication
(10) Pub. No.: US 2025/0255592 A1
(43) Pub. Date: Aug. 14, 2025

(54) JAW ASSEMBLY FOR MEDICAL DEVICES AND METHODS OF USING THE SAME

(71) Applicant: Boston Scientific Scimed, Inc., Maple Grove, MN (US)

(72) Inventors: Marco Vinicio ARIAS ALPIZAR, San José (CR); Mauro VÍQUEZ GONZALEZ, San Isidro (CR); Gonzalo Jose SAENZ VILLALOBOS, Heredia (CR); Esteban de la Trinidad JIMÉNEZ SÁNCHEZ, San Jose (CR); Gabriela GAMBOA BASTOS, San Jose (CR); Viviana ARTAVIA SALAS, Heredia (CR); Rafael BERENZON, La Union (CR); Oscar SEGURA ELIZONDO, San Jose (CR); Gustavo CARVAJAL BONILLA, Flores (CR); Fernando VALERIO GUERRERO, Alajuela (CR); VALERIA ZÚÑIGA VARGAS, San Francisco (CR)

(73) Assignee: Boston Scientific Scimed, Inc., Maple Grove, MN (US)

(21) Appl. No.: 19/050,667

(22) Filed: Feb. 11, 2025

Related U.S. Application Data
(60) Provisional application No. 63/552,293, filed on Feb. 12, 2024.

(51) Int. Cl. A61B 10/06; A61B 10/04

(52) U.S. Cl. CPC A61B 10/06; A61B 10/04

(57) ABSTRACT

A medical device that includes a wire assembly including an impediment on a distal portion of the wire assembly, a jaw assembly coupled to the wire assembly with the impediment disposed inside the jaw assembly, and a lever movably coupled to the distal portion and disposed inside the jaw assembly. The jaw assembly is configured to move between a closed state and an opened state. The lever is configured to contact an interior of the jaw assembly. The wire assembly is configured to move the jaw assembly from the closed state towards the opened state in response to the wire assembly translating distally inside the jaw assembly and moving the lever proximally until engaging the impediment. The lever is configured to pivot outwards relative to the jaw assembly upon engaging the impediment, and push against the interior of the jaw assembly, thereby urging the jaw assembly towards the opened state.

### CROSS-REFERENCE TO RELATED APPLICATIONS

This application claims the benefit of priority of U.S. Provisional Patent Application No. 63/552,293, filed on Feb. 12, 2024, the entirety of which is incorporated herein by reference.

### TECHNICAL FIELD

Various aspects of this disclosure relate generally to medical devices including jaw assemblies for attachment onto medical systems or devices, and related methods. For example, the disclosure includes devices, systems, and related methods for extracting objects (e.g., a tissue sample) from a subject with a medical device including a jaw assembly attached over a shaft of a medical system.

### BACKGROUND

In a medical procedure, an operator may insert a medical system, such as an endoscope or other type of scope, into a body lumen of a subject (e.g., a patient) to access a procedure site. To extract a target object (e.g., a tissue sample) from the procedure site, a medical instrument may extend through a channel of the endoscope and exit from a distal end of the endoscope to obtain the target object. A size of the medical instrument, such as a pair of biopsy jaws, may dictate a size of the target object that may be extracted from the procedure site.

However, the size of the medical instrument may be restricted by a size and/or a shape of the channel or distal end of the endoscope that the medical instrument is received through, thereby limiting the potential size of the target object that the medical instrument is capable of extracting. As a diagnostic yield of a tissue sample is proportionately greater with a size of the tissue sample that is extracted, a need exists for systems, devices, and/or methods that expand tissue sample extraction capabilities of medical devices.

### SUMMARY

Aspects of the disclosure relate to, among other things, systems, devices, and methods for a medical device including a jaw assembly attached over a shaft of a medical system or device (e.g., an endoscope). The jaw assembly may facilitate illumination, imaging, and access to a procedure site for extraction of a target object (e.g., a tissue sample), among other aspects. Each of the aspects disclosed herein may include one or more of the features described in connection with any of the other disclosed aspects.

According to an example, a medical device includes a wire assembly including an impediment on a distal portion of the wire assembly; a jaw assembly coupled to the wire assembly with the impediment disposed inside the jaw assembly, wherein the jaw assembly is configured to move between a closed state and an opened state; and a lever movably coupled to the distal portion and disposed inside the jaw assembly, wherein the lever is configured to contact an interior of the jaw assembly; wherein the wire assembly is configured to move the jaw assembly from the closed state towards the opened state in response to the wire assembly translating distally inside the jaw assembly and moving the lever proximally until the lever engages the impediment; wherein the lever is configured to pivot outwards relative to the jaw assembly upon engaging the impediment and push against the interior of the jaw assembly, thereby urging the jaw assembly towards the opened state.

Any of the medical devices described herein may include any of the following features. The wire assembly includes an engagement mechanism on the distal portion such that the engagement mechanism is disposed inside the jaw assembly in at least some configurations of the jaw assembly. The engagement mechanism is configured to translate distally relative to the jaw assembly from a proximal position to a distal position in response to the wire assembly translating distally. The engagement mechanism is disposed inside the jaw assembly when in the proximal position, and extends at least partially outside of the jaw assembly when in the distal position. The wire assembly is configured to rotate relative to the jaw assembly, and the engagement mechanism is configured to rotate in response to rotation of the wire assembly. The engagement mechanism is configured to pierce a tissue positioned adjacent to the jaw assembly in response to the wire assembly translating distally when the jaw assembly is in the opened state. The engagement mechanism is configured to securely engage the tissue in response to the wire assembly rotating relative to the jaw assembly. The engagement mechanism is configured to pull the tissue proximally into the jaw assembly in response to the wire assembly translating proximally relative to the jaw assembly. The jaw assembly is configured to cut the tissue engaged by the engagement mechanism and received inside the jaw assembly in response to the wire assembly translating proximally, thereby causing the jaw assembly to move from the opened state towards the closed state. The engagement mechanism includes a needle, a corkscrew, a hook, or a barbed spike. The wire assembly is configured to translate proximally relative to the jaw assembly and the lever is configured to translate distally relative to the distal portion. The impediment is configured to disengage the lever in response to the lever translating distally, such that continued translation proximal of the wire assembly is configured to pivot the lever inwards within the jaw assembly. The lever is configured to move the jaw assembly towards one another when pivoting inwards, thereby moving the jaw assembly from the opened state towards the closed state. The wire assembly extends through a shaft of an endoscope with the distal portion extending outwardly from a distal tip of the shaft, and the jaw assembly is coupled over the distal tip. The jaw assembly includes an imaging channel aligned with an imaging mechanism on the distal tip and a pair of lighting channels aligned with a pair of lighting mechanisms on the distal tip; and wherein, with the jaw assembly coupled over the distal tip, the imaging mechanism is configured to generate images distally from the jaw assembly through the imaging channel and the pair of lighting mechanisms are configured to emit light distally from the jaw assembly through the pair of lighting channels.

According to another example, a medical device includes a wire assembly including an impediment on a distal portion of the wire assembly; a jaw assembly coupled to the wire assembly with the impediment disposed inside the jaw assembly, wherein the jaw assembly includes a pair of jaws configured to move between a closed state and an opened state; and a lever movably coupled to the distal portion and disposed inside the jaw assembly, wherein the lever is configured to contact at least one of the pair of jaws; wherein the wire assembly is configured to translate along a distal direction relative to the jaw assembly and the lever is configured to translate simultaneously towards a proximal direction relative to the distal portion; and wherein the impediment is configured to engage the lever and inhibit further translation of the lever towards the proximal direction, such that continued translation of the wire assembly along the distal direction is configured to pivot the lever outwards from within the jaw assembly, and the lever is configured to move the at least one of the pair of jaws away from the other one of the pair of jaws when pivoting within the jaw assembly, thereby moving the pair of jaws from the closed state towards the opened state.

Any of the medical devices described herein may include any of the following features. The wire assembly includes an engagement mechanism on the distal portion that is configured to translate towards the distal direction in response to the wire assembly translating towards the distal direction, and rotate in response to the wire assembly rotating relative to the jaw assembly. The engagement mechanism is configured to pierce a tissue positioned adjacent to the jaw assembly in response to the wire assembly translating towards the distal direction when the jaw assembly is in the opened state, and securely engage the tissue in response to the wire assembly rotating relative to the jaw assembly. The engagement mechanism is configured to pull the tissue proximally into the jaw assembly in response to the wire assembly translating towards the proximal direction relative to the jaw assembly; and wherein the jaw assembly is configured to cut the tissue engaged by the engagement mechanism in response to the wire assembly translating towards the proximal direction, thereby causing the pair of jaws to move from the opened state towards the closed state.

According to a further example, a medical system includes a shaft including a working channel and a distal tip; a wire assembly movably disposed inside the working channel, the wire assembly including a distal portion extending outwardly from the distal tip; a jaw assembly coupled to the wire assembly and positioned over the distal tip such that the jaw assembly abuts against a distalmost face of the distal tip, wherein the jaw assembly is configured to move between a closed state and an opened state; and a lever movably coupled to the distal portion and disposed inside the jaw assembly and outside of the shaft, wherein the lever is configured to contact the jaw assembly; wherein the wire assembly is configured to move the jaw assembly distally away from the distal tip in response to a distal translation relative to the shaft, and move the lever proximally within the jaw assembly in response to further distal translation relative to the shaft; and wherein the lever is configured to pivot outwards within the jaw assembly to push against the jaw assembly, thereby moving the jaw assembly from the closed state towards the opened state.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1A depicts a perspective view of a proximal end of a medical system including a handle and an insertion portion, according to aspects of this disclosure.

FIG. 1B depicts a perspective view of a distal end of the medical system of FIG. 1A including a distal tip of the insertion portion, according to aspects of this disclosure.

FIG. 2 depicts a perspective view of an exemplary medical device coupled over the distal end of the medical system of FIG. 1B, the medical device positioned in a closed state, according to aspects of this disclosure.

FIG. 3 depicts a perspective view of the medical device of FIG. 2 positioned in an opened state, according to aspects of this disclosure.

FIG. 4 depicts a partial perspective view of the medical device of FIG. 2, according to aspects of this disclosure.

FIG. 5 depicts a perspective view of a handle assembly of the medical device of FIG. 2, according to aspects of this disclosure.

FIG. 6 depicts a perspective view of the medical device of FIG. 2 in an extended position relative to the distal end of the medical system of FIG. 1B, according to aspects of this disclosure.

FIG. 7 depicts a partial side view of the medical device of FIG. 2 moving from a closed state to an opened state, according to aspects of this disclosure.

FIG. 8A depicts a perspective view of another exemplary medical device coupled to the distal end of the medical system of FIG. 1B, the medical device positioned in a closed state, according to aspects of this disclosure.

FIG. 8B depicts a perspective view of the medical device of FIG. 8A positioned in an opened state, according to aspects of this disclosure.

### DETAILED DESCRIPTION

This disclosure relates, in certain aspects, to medical devices including a jaw assembly coupled over a shaft of a medical system or device (e.g., an endoscope) for facilitating acquisition of a target object (e.g., a tissue sample) from a procedure site in a subject (e.g., patient). For example, medical systems may be equipped with a medical device, and particularly a jaw assembly, that includes components, devices, and/or mechanisms that are arranged relative to various channels of a shaft of the medical system that facilitate illuminating, imaging, accessing, and treating the procedure site. The jaw assembly may be movable (e.g., translatable) relative to the shaft of the medical system from a first (proximal) position to a second (distal) position. The jaw assembly may be automatically movable (e.g., pivotable) between a closed state and an opened state in response to the jaw assembly moving from the first (proximal) position to the second (distal) position.

In examples, accessing a procedure site may include endoluminal placement of the medical device into the patient, such as through an anatomical passageway via a natural orifice. The orifice can be, for example, the nose, mouth, or anus, and the placement can be in any portion of the GI tract, including the esophagus, stomach, duodenum, large intestine, or small intestine. Placement also can be in other organs or other bodily spaces reachable via the GI tract, other body lumens, or openings in the body. This disclosure is not limited to any particular medical procedure or treatment site within a body.

Examples of the disclosure may relate to devices and methods for performing various medical procedures and/or treating portions of the large intestine (colon), small intestine, cecum, esophagus, pancreatic-biliary tract, any other portion of the gastrointestinal tract, and/or any other suitable patient anatomy (collectively referred to herein as a “target treatment site”). As mentioned above, this disclosure is not limited to any specific medical device or method, and aspects of the disclosure may be used in connection with any suitable medical tool and/or medical method, at any suitable site within the body.

Reference will now be made in detail to aspects of the disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the subject. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

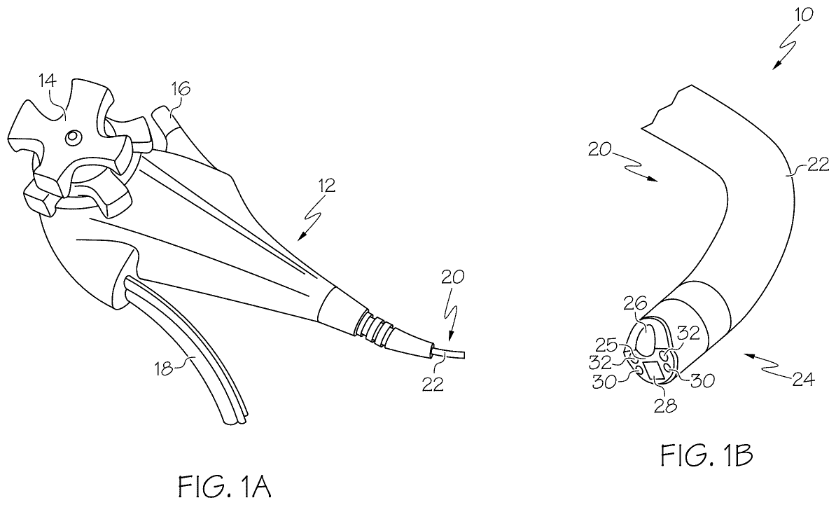

FIGS. 1A-1B depict aspects of an exemplary medical device or system 10. Medical system 10 may include an insertion device, such as an endoscope, which may be inserted into an esophagus of a patient. FIG. 1A depicts a proximal portion of medical system 10 including a handle 12 for gripping and operation by a user, and an insertion portion 20 extending distally from handle 12 for at least partial insertion into a subject (e.g., a patient). Handle 12 may include one or more actuators 14, for example, on a proximal portion of handle 12. The one or more actuators 14 may be configured and operable to facilitate articulation and/or steering of insertion portion 20, including a distal tip 24 of insertion portion 20 (see FIG. 1B). The one or more actuators 14 may include one or more knobs, buttons, sliders, joysticks, or various other suitable actuators. It will be appreciated that medical system 10 may include any suitable number of actuators 14 on handle 12.

Handle 12 may include a port 16 that is configured and operable to provide access to one or more channels of medical system 10, such as a working channel extending through handle 12 and insertion portion 20. For example, a medical instrument or other device may be coupled to and/or inserted into medical system 10 via port 16. The working channel may extend longitudinally through a length of insertion portion 20, such that the medical instrument received through port 16 may extend through the working channel and exit insertion portion 20 at distal tip 24 (FIG. 1B). Medical system 10 may include an umbilicus 18 that extends from handle portion 12, and umbilicus 18 may include one or more wires, cables, and/or conduits for providing, power, signals, and/or fluids to or from handle 12. For example, umbilicus 18 may be configured and operable to connect handle 12 to one or more user interfaces, monitors, displays, and/or control systems.

Insertion portion 20 may include a shaft 22 extending distally from handle 12. Shaft 22 may be flexible and include one or more wires, tubes, channels, and/or other features passing therethrough. Shaft 22 may terminate at distal tip 24, as depicted in FIG. 1B, which may be positioned at a procedure site within the subject during use. As shown in FIG. 1B, distal tip 24 may include a distalmost face 25 that defines a working channel opening 26. The working channel extending through handle 12 and shaft 20 may terminate at working channel opening 26, such that the one or more medical instruments or other devices received through port 16 may extend out of the working channel via working channel opening 26. As described herein, the medical instrument extending distally outwards from working channel opening 26 may be used to perform a medical procedure on the subject, such as extraction of a target object (e.g., a tissue sample) from the procedure site.

Still referring to FIG. 1B, medical system 10 may include one or more devices and/or systems at distal tip 24, such as an imaging mechanism 28, a pair of lighting mechanisms 30, and a pair of irrigation openings 32. In the example, imaging mechanism 28 may include an image sensor and/or a camera, such as a ball grid array (“BGA”)-style camera. Imaging mechanism 28 may be configured to generate a video and/or images in a distal direction from distal tip 24. For example, imaging mechanism 28 may be configured and operable to provide a signal to a remote monitor (not shown), such that a user of medical system 10 may view a video or image display generated by imaging mechanism 28 while navigating medical system 10 through a body of the subject and performing the medical procedure (e.g., a tissue sample extraction).

Each of the pair of lighting mechanisms 30 may include one or more (e.g., two) light emitting diodes (“LEDs”), fiber optic light guides, or various other suitable light sources. Each of the pair of irrigation openings 32 may be in fluid communication with one or more irrigation channels extending through handle 12 and insertion portion 20, such that the irrigation channel(s) may terminate at irrigation openings 32. In this instance, the pair of irrigation openings 32 may be configured and operable to irrigate a surrounding environment of distal tip 24 when shaft 22 is positioned within the subject and adjacent to the procedure site. In some examples, the one or more irrigation channels extending through shaft 22 and handle 12 may be fluidly coupled to a remote device and/or system via umbilicus 18 (see FIG. 1A).

Although a single imaging mechanism 28, a pair of lighting mechanisms 30, and a pair of irrigation openings 32 are depicted in the example, it should be appreciated that additional imaging mechanisms 28, and additional and/or fewer lighting mechanisms 30 and irrigation openings 32, may be included on distal tip 24 without departing from a scope of this disclosure. Alternatively, in other examples, imaging mechanism 28 and lighting mechanisms 30 may be combined into a single device.

As depicted in FIG. 1B, medical system 10 may be “forward-facing.” In other words, features of distal tip 24 (e.g., working channel opening 26, imaging mechanism 28, lighting mechanisms 30, and irrigation openings 32) may face distally (i.e., forward of distalmost face 25). It should be appreciated that aspects of this disclosure also encompass other configurations of distal tip 24. For example, medical system 10 may be “side-facing” in which one or more of working channel opening 26, imaging mechanism 28, lighting mechanisms 30, and/or irrigation openings 32 may be disposed on a radially outer side of distal tip 24. In this instance, such openings and/or mechanisms may face and/or point in a radially outward direction, such as approximately perpendicularly to a longitudinal axis of insertion portion 20.

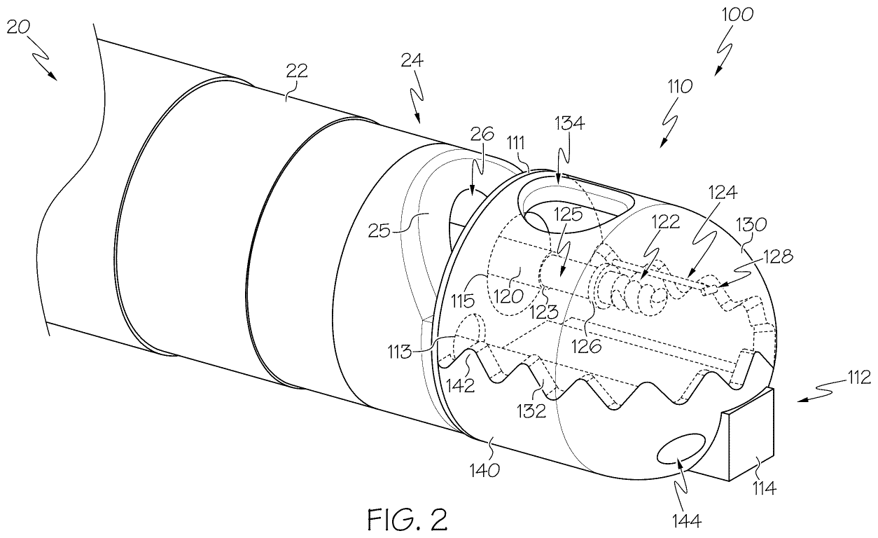

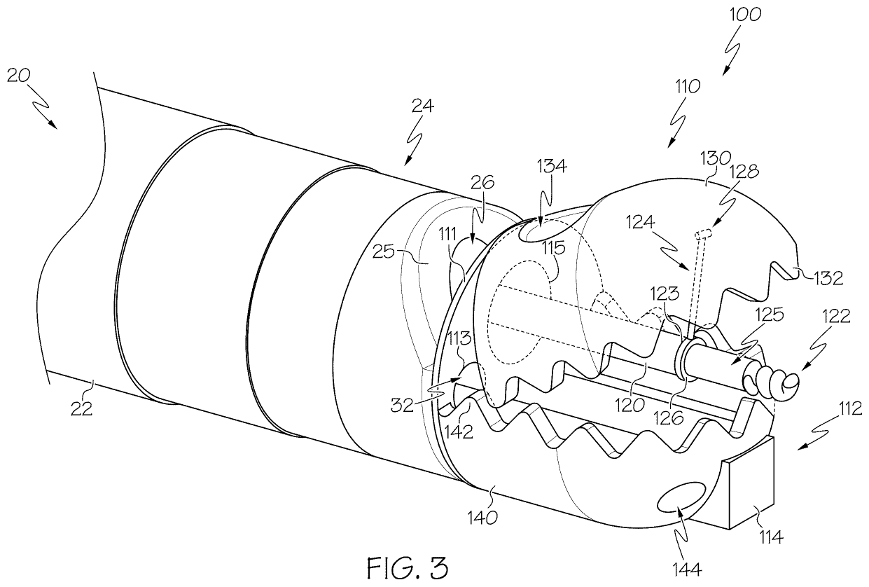

Referring now to FIGS. 2-3, an exemplary medical device 100 is depicted. Medical device 100 may include one or more assemblies coupled to one or more components of medical system 10, such as handle 12, shaft 22, and distal tip 24. For example, the one or more assemblies of medical device 100 may be received through the working channel of handle 12 and shaft 22, and one or more additional assemblies of medical device 100 may be coupled over distal tip 24, such that said assemblies of medical device 100 may extend distally from distalmost face 25.

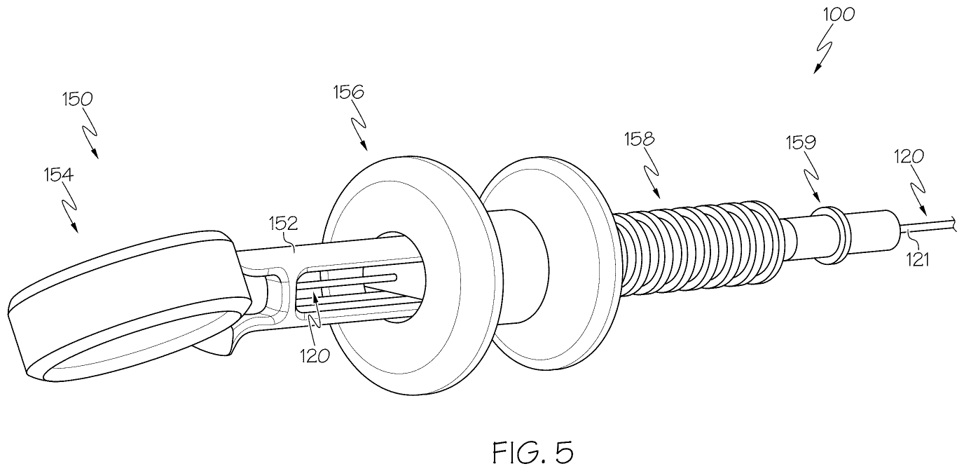

In the example, medical device 100 may include a jaw assembly 110 positioned distally and/or over distal tip 24, a wire assembly 120 movably disposed through a working channel of handle 12 and shaft 22, and a handle assembly 150 (see FIG. 5) coupled to a proximal portion of wire assembly 120 adjacent to handle 12. Wire assembly 120 may be disposed between and coupled to each of jaw assembly 110 and handle assembly 150. As described herein, handle assembly 150 may be configured and operable to control movement of jaw assembly 110 via wire assembly 120.

Jaw assembly 110 may be disposed adjacent to distal tip 24 and positioned at least partially against distalmost face 25. In some embodiments, jaw assembly 110 may be at least partially disposed over a portion of distal tip 24, and particularly distalmost face 25. With jaw assembly 110 positioned distally of and/or over distal tip 24, a distal face 112 of jaw assembly 110 may be disposed distally of distalmost face 25. Handle assembly 150 (FIG. 5) may be positioned adjacent to handle 12 (FIG. 1A) for controlling jaw assembly 110, with wire assembly 120 received by port 16 and extending through the working channel of handle 12 and shaft 22.

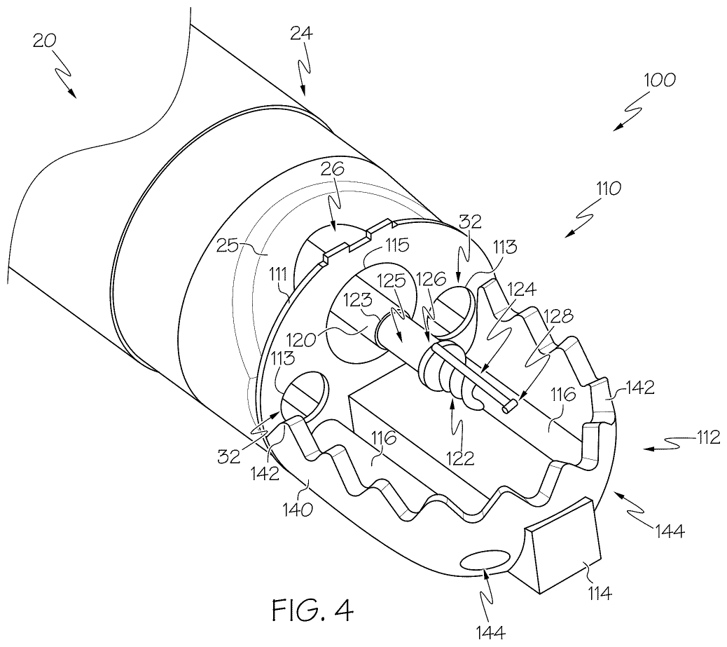

Still referring to FIGS. 2-3, jaw assembly 110 may include a face plate 111, an imaging channel 114 and a pair of lighting channels 116 extending distally from face plate 111 (see FIG. 4), and a pair of jaws including a first (upper) jaw 130 and a second (lower) jaw 140 coupled to face plate 111. Face plate 111 may be positioned proximal to the pair of jaws 130, 140. Imaging channel 114 and the pair of lighting channels 116 may each include enclosed lumens that extend distally from face plate 111 and through lower jaw 140. For example, imaging channel 114 may be sized, shaped, and positioned relative to lower jaw 140 to align with imaging mechanism 28 when jaw assembly 110 is coupled over distal tip 24, and particularly with face plate 111 disposed over distalmost face 25. In the example, imaging channel 114 may extend at least partially outwards from lower jaw 140 in a distal direction, such as distally from distal face 112 of jaw assembly 110. For example, imaging channel 114 may extend approximately parallel to a central longitudinal axis of jaw assembly 110.

In this instance, imaging channel 114 may be configured and operable to provide use of imaging mechanism 28 through lower jaw 140, and particularly allow imaging in a distal direction from distal face 112 when jaw assembly 110 is coupled over distalmost face 25 of distal tip 24. In other words, imaging channel 114 may be operable to extend imaging mechanism 28 from distal tip 24 through jaw assembly 110. In some embodiments, imaging channel 114 may include a glass substrate, a clear lens, and/or various other suitable structures for extending imaging mechanism 28 from distal tip 24 through jaw assembly 110, and particularly lower jaw 140. In other embodiments, imaging channel 114 may terminate at distal face 112 such that imaging channel 114 does not extend distally outwards from lower jaw 140.

As best seen in FIG. 4, lighting channels 116 may be sized, shaped, and positioned relative to lower jaw 140 to align with lighting mechanisms 30 when jaw assembly 110 is coupled to distal tip 24 with face plate 111 disposed over distalmost face 25. Lighting channels 116 may be entirely disposed within lower jaw 140. As described herein, lower jaw 140 may include one or more openings 144 aligned with lighting channels 116 to allow a light emitted by lighting mechanisms 30 to extend through lighting channels 116 and distally outwards from jaw assembly 110, and particularly lower jaw 140, via openings 144. In this instance, lighting channels 116 may be configured and operable to provide use of lighting mechanisms 30 through lower jaw 140, and particularly allow for illumination in a distal direction from distal face 112 when jaw assembly 110 is coupled to distal tip 24 with face plate 111 disposed over distalmost face 25. In other words, lighting channels 116 may be operable to extend lighting mechanisms 30 from distal tip 24 through jaw assembly 110.

Referring back to FIGS. 2-3, lower jaw 140 may include one or more openings 144 positioned along distal face 112 of jaw assembly 110. The one or more openings 144 may be aligned with corresponding lighting channels 116 extending through lower jaw 140. Each of lighting channels 116 may be coupled to a respective lighting mechanism 30 at distal tip 24. As such, lighting channels 116 and openings 144 may be configured and operable to facilitate uninterrupted use of lighting mechanisms 30 through jaw assembly 110 despite medical device 100 being coupled to distal tip 24 and positioned over distalmost face 25.

In the example, and as best seen in FIG. 4, jaw assembly 110 may include a pair of lighting channels 116 and lower jaw 140 may include a pair of openings 144 corresponding to the pair of lighting mechanisms 30 on distal tip 24. It should be appreciated that a quantity, a size, and a location of lighting channels 116 and openings 144 may be adjusted based on the corresponding properties of lighting mechanisms 30 on distal tip 24.

Still referring to FIGS. 2-3, face plate 111 may be disposed between distalmost face 25 and the pair of jaws 130, 140 when jaw assembly 110 is coupled to distal tip 24. It should be appreciated that jaw assembly 110 is sized and/or shaped such that the pair of jaws 130, 140 cannot pass through the working channel of handle 12 and shaft 22 of insertion portion 20. As such, jaw assembly 110 is disposed over distal tip 24. Upper jaw 130 may include a plurality of teeth 132 and lower jaw 140 may include a plurality of teeth 142. Upper jaw 130 and lower jaw 140 may be configured to mate with one another, with the plurality of teeth 132 engaging (e.g., interdigitating with) the corresponding plurality of teeth 142 when jaw assembly 110 is in a closed state, as seen in FIG. 2. As described herein, jaw assembly 110 may be configured to extract a target object (e.g., a tissue sample) from the procedure site within the subject (e.g., a patient) in response to the plurality of teeth 132, 142 severing and/or separating the target object from a surrounding environment (e.g., tissue) of the procedure site.

In the example, upper jaw 130 may be configured to move relative to face plate 111 and/or lower jaw 140 to interchangeably transition jaw assembly 110 between the closed state (FIG. 2) and an opened state (FIG. 3). In this instance, lower jaw 140 may remain fixed relative to upper jaw 130, such that lower jaw 140 is immovable. In other embodiments, lower jaw 140 may be configured to move in lieu of and/or simultaneously with upper jaw 130, such as relative to face plate 111. An upper surface of upper jaw 130 may include an opening 134 that is sized, shaped, and/or otherwise configured to allow one or more residual materials (e.g., fluid) to exit upper jaw 130 when jaw assembly 110 is moving from the opened state to the closed state, such as to release the residual material and maintain sufficient volume within an interior cavity of jaw assembly 110 for receiving the target object during extraction.

Jaw assembly 110 (e.g., face plate 111) may include a pair of irrigation apertures 113 that are positioned in and/or on face plate 111 to align with the pair of irrigation openings 32 when jaw assembly 110 is coupled to distal tip 24 with face plate 111 disposed over distalmost face 25. Jaw assembly (e.g., face plate 111) 110 may further include a working aperture 115 that is positioned in and/or on face plate 111 to align with working channel opening 26 when jaw assembly 110 is coupled to distal tip 24.

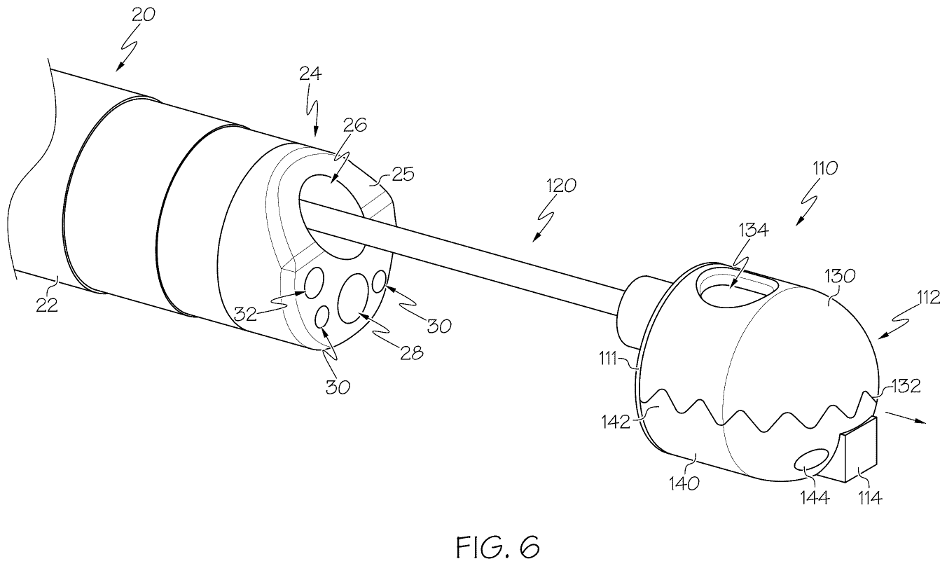

Still referring to FIGS. 2-4, a distal portion 125 of wire assembly 120 may extend out of the working channel of shaft 22 via working channel opening 26 and into jaw assembly 110 via working aperture 115 on face plate 111. As described herein, jaw assembly 110 may be configured to move (e.g., translate) relative to distal tip 24 from a first (proximal) position (FIG. 2) to a second (distal) position (FIG. 6) in response to actuation of handle assembly 150 (FIG. 5). Jaw assembly 110 may be further configured to move (e.g., pivot) upper jaw 130 relative to lower jaw 140 from the closed state (FIG. 2) to the opened state (FIG. 3) in response to further actuation of handle assembly 150.

Wire assembly 120 may include an engagement mechanism 122 at distal portion 125 and a movable lever 124 slidably coupled to distal portion 125. Movable lever 124 may be positioned proximal to engagement mechanism 122. Engagement mechanism 122 may be sized, shaped, and/or otherwise configured to engage the target object (e.g., a tissue sample) that is received within jaw assembly 110 prior to the pair of jaws 130, 140 separating the target object from the surrounding environment (e.g. tissue) at the procedure site. In the example, engagement mechanism 122 may include a needle, a corkscrew, a hook, a barbed spike, and/or various other suitable devices for grasping, piercing, puncturing, and/or attaching wire assembly 120 to the target object. As described herein, engagement mechanism 122 may be configured to pierce the target object in response to translating towards the target object, and intertwine the target object onto the wire assembly in response to rotating inside and/or adjacent to the target object.

Still referring to FIGS. 2-4, movable lever 124 may be slidably coupled to distal portion 125 of wire assembly 120, with distal portion 125 having a longitudinal length defined between an impediment 123 on wire assembly 120 and engagement mechanism 122. Impediment 123 may define a ledge, a protrusion, an abutment, a stop, a step, and/or another structural impediment formed along an exterior surface of wire assembly 120 at a proximal end of distal portion 125. Impediment 123 may define a proximalmost position that movable lever 124 may move (e.g., translate) along distal portion 125 of wire assembly 120. In other words, impediment 123 may be configured to engage movable lever 124, thereby inhibiting proximal translation of movable lever 124 relative to distal portion 125 beyond the proximalmost position defined by impediment 123. As described herein, impediment 123 may be configured to disengage movable lever 124 in response to a distal translation of movable lever 124 relative to distal portion 125.

Engagement mechanism 122 may define an opposing, distalmost position that movable lever 124 may move along distal portion 125 of wire assembly 120. In other words, a proximal end of engagement mechanism 122 may define a corresponding protrusion, abutment, stop, and/or impediment that is positioned at a distal (opposite) end of distal portion 125 from impediment 123. In this instance, engagement mechanism 122 may be configured to engage movable lever 124, thereby inhibiting distal translation of movable lever 124 relative to distal portion 125 beyond the distalmost position defined by engagement mechanism 122. In some embodiments, wire assembly 120 may include a second impediment at the distal end of distal portion 125 (and/or proximal end of engagement mechanism 122) that is substantially similar to impediment 123 shown and described above.

Movable lever 124 may include a proximal ring 126 and a distal leg 128 positioned opposite of proximal ring 126, with a longitudinal length of movable lever 124 defined between proximal ring 126 and distal leg 128. Proximal ring 126 may include an opening that is sized, shaped, and/or otherwise configured to receive distal portion 125 of wire assembly 120, thereby slidably coupling movable lever 124 to wire assembly 120. Distal leg 128 may include a peg, a protrusion, an abutment end, a tab, and/or various other suitable interfaces that is sized, shaped, and/or otherwise configured to abut against an interior surface of upper jaw 130. In some embodiments, distal leg 128 may be in continuous contact with the interior surface of upper jaw 130 when jaw assembly 110 is in the closed state (FIG. 2) and the opened state (FIG. 3). In other embodiments, distal leg 128 may be configured to disengage the interior surface of upper jaw 130 when jaw assembly 110 is in the closed state.

Movable lever 124 may be configured to move jaw assembly 110 from the closed state to the opened state in response to distal leg 128 urging (e.g., pushing) upper jaw 130 outwards (e.g., upwards) relative to lower jaw 140 as proximal ring 126 moves (e.g., translates) proximally away from engagement mechanism 122 and towards impediment 123 along distal portion 125. In other words, jaw assembly 110 may be configured to move from the closed state to the opened state in response to movable lever 124 (e.g., proximal ring 126 of movable lever 124) translating proximally along wire assembly 120 from the first (proximal) position (with proximal ring 126 positioned adjacent to engagement mechanism 122 relative to impediment 123) to the second (distal) position (with proximal ring 126 positioned adjacent to impediment 123 relative to engagement mechanism 122). In this instance, movable lever 124 (e.g., distal leg 128 of movable lever 124) may pivot outwards (e.g., upwards) relative to proximal ring 126, thereby causing distal leg 128 to apply a force against upper jaw 130 in an outwards (upwards) direction to open jaw assembly 110.

Movable lever 124 may be further configured to move jaw assembly 110 from the opened state to the closed state in response to distal leg 128 ceasing to urge (e.g., push) upper jaw 130 outwards (upwards) relative to lower jaw 140 as proximal ring 126 moves (e.g., translates) distally towards engagement mechanism 122 and away from impediment 123 along distal portion 125. Stated differently, jaw assembly 110 may be configured to move from the opened state to the closed state in response to movable lever 124 (e.g., proximal ring 126 of movable lever 124) translating distally along wire assembly 120 from the second (distal) position to the first (proximal) position. In this instance, movable lever 124 (e.g., distal leg 128 of movable lever 124) may pivot inwards (e.g., downwards) relative to proximal ring 126, thereby causing distal leg 128 to cease application of the force against upper jaw 130 in the outwards (upwards) direction to close jaw assembly 110.

As best seen in FIG. 4, irrigation channels 32 on distal tip 24 are not covered and/or obstructed by upper jaw 130 and lower jaw 140 when jaw assembly 110 is coupled to distal tip 24 with face plate 111 disposed over distalmost face 25. As such, irrigation channels 32 are exposed to an interior cavity of jaw assembly 110, collectively defined between upper jaw 130 and lower jaw 140, via irrigation apertures 113 on face plate 111. As such, irrigation channels 32 may be configured and operable to provide irrigation in a distal direction from distal tip 24 despite jaw assembly 110 being coupled to distal tip 24 and face plate 111 being disposed over distalmost face 25.

FIG. 5 depicts handle assembly 150 of medical device 100. Handle assembly 150 may include a main body 152, a first actuator 154 rotatably coupled to main body 152, a second actuator 156 slidably coupled to main body 152, a distal portion 158, and a coupler 159 along distal portion 158. Handle assembly 150 may be coupled to wire assembly 120 such that actuation of first actuator 154 and/or second actuator 156 may provide a corresponding movement of wire assembly 120. In the example, a proximal portion 121 of wire assembly 120 may be releasably coupled to handle assembly 150 via coupler 159. Accordingly, handle assembly 150 may be configured and operable to selectively decouple wire assembly 120 by detaching proximal portion 121 from coupler 159. In some embodiments, wire assembly 120 may extend through distal portion 158 and main body 152 with each of first actuator 154 and second actuator 156 coupled to at least a portion of wire assembly 120.

In the example, first actuator 154 may be configured and operable to rotate wire assembly 120 in response to a rotation of first actuator 154 relative to main body 152. In this instance, by rotating proximal portion 121 of wire assembly 120, the opposite distal portion 125 of wire assembly 120 may simultaneously rotate, thereby causing rotation of engagement mechanism 122 (see FIG. 4). In this instance, upon moving jaw assembly 110 to the opened state and positioning engagement mechanism 122 adjacent to and/or against the target object, first actuator 154 may be configured to rotate engagement mechanism 122 into the target object, thereby causing engagement mechanism 122 to securely engage the target object. In other words, first actuator 154 may be configured to puncture and/or screw the target object with engagement mechanism 122 via rotation of first actuator 154 relative to main body 152. It should be appreciated that with distal portion 125 extending through the opening of proximal ring 126, wire assembly 120 may rotate relative to proximal ring 126 without causing movable lever 124 to rotate.

Still referring to FIG. 5, second actuator 156 may be configured and operable to translate wire assembly 120 in response to a translation of second actuator 156 relative to main body 152, such as in a proximal direction and a distal direction. In this instance, by translating proximal portion 121 of wire assembly 120 in the proximal and/or distal direction, the opposite distal portion 125 of wire assembly 120 may simultaneously translate in the same direction, thereby causing a corresponding movement of engagement mechanism 122 (see FIG. 4) in the same direction.

As seen in FIG. 6, second actuator 156 may be configured to translate wire assembly 120 distally relative to distal tip 24, thereby detaching face plate 111 from distalmost face 25 and jaw assembly 110 from distal tip 24 of shaft 22. In this instance, a user may position jaw assembly 110 adjacent to the target object by actuating second actuator 156 and moving jaw assembly 110 away from distal tip 24 and towards the target object. It should be appreciated that movable lever 124 may be configured to translate proximally relative to wire assembly 120 and/or engagement mechanism 122 as wire assembly 120 and engagement mechanism 122 translate in the distal direction due to distal leg 128 abutting against the interior surface of upper jaw 130. Stated differently, movable lever 124 may move in an opposite direction of wire assembly 120 and/or engagement mechanism 122 until proximal ring 126 engages impediment 123.

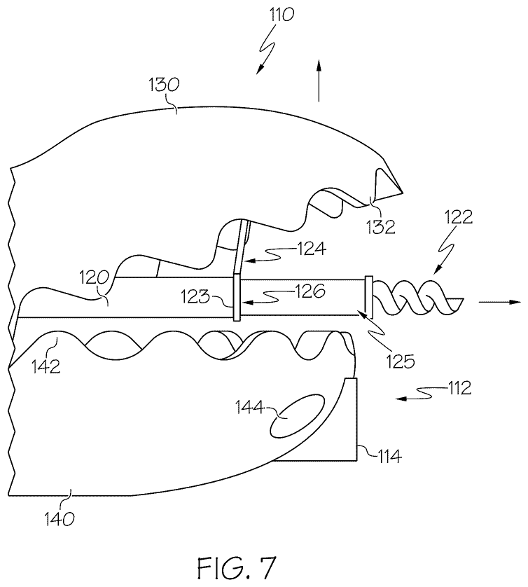

In this instance, as seen in FIG. 7, second actuator 156 may be configured to transition jaw assembly 110 from the closed state to the opened state in response to a further distal translation of second actuator 156 relative to main body 152. With proximal ring 126 engaged against impediment 123 and distal leg engaged with upper jaw 130, movable lever 124 may be configured to pivot outwards (upwards) relative to proximal ring 126 as wire assembly 120 continues to translate distally from the corresponding distal translation of second actuator 156. Distal leg 128 may be configured to pivot upper jaw 130 outwards (upwards) away from lower jaw 140 as movable lever 124 pivots outwards (upwards) relative to proximal ring 126, thereby opening jaw assembly 110 towards the opened state. With jaw assembly 110 moved to the opened state, engagement mechanism 122 may be exposed from within the internal cavity between upper jaw 130 and lower jaw 140 for attachment with the target object.

In exemplary use, medical device 100 may be coupled to medical system 10 with handle assembly 150 positioned adjacent to handle 12 and wire assembly 120 extending proximally through working channel opening 26 and into the working channel of insertion portion 20 and handle 12 until exiting port 16. In other words, wire assembly 120 may be back-loaded into medical system 10 via working channel opening. Proximal portion 121 of wire assembly 120 may be coupled to handle assembly 150 via coupler 159. After medical device 100 is coupled to medical system 10, distal portion 125 of wire assembly 120 may be positioned outside of working channel opening 26 of distal tip 24 with jaw assembly 110 disposed over distalmost face 25 of distal tip 24. In this instance, wire assembly 120, and particularly engagement mechanism 122 and distal portion 125, may be disposed within the internal cavity defined between upper jaw 130 and lower jaw 140.

Imaging mechanism 28 and lighting mechanisms 30 may facilitate navigation of insertion portion 20 through one or more bodily lumens of the subject as medical system 10 is moved towards the procedure site. Despite jaw assembly 110 being disposed over distalmost face 25 of distal tip 24, medical device 100 may be configured to allow imaging mechanism 28 to provide a visual navigation of insertion portion 20 within the subject via imaging channel 114. Stated differently, imaging channel 114 may be aligned with imaging mechanism 28 when jaw assembly 110 is coupled over distal tip 24, such that imaging mechanism 28 may continue to generate a visual display of the surrounding environment of distal tip 24 through jaw assembly 110 via imaging channel 114.

Additionally, medical device 100 may be configured to allow lighting mechanisms 30 to illuminate the surrounding environment of distal tip 24 via lighting channels 116. In other words, lighting channels 116 may be aligned with lighting mechanisms 30 when jaw assembly 110 is coupled over distal tip 24, such that lighting mechanisms 30 may continue to emit light outwards towards the surrounding environment of distal tip 24 through jaw assembly 10 via lighting channels 116. Insertion portion 20 may be navigated through the subject and distal tip 24 may be positioned at the procedure site via actuation of the one or more actuators 14 on handle 12 (FIG. 1A).

In this instance, second actuator 156 of handle assembly 150 may be moved distally relative to main body 152 to extend jaw assembly 110 and wire assembly 120 distally relative to distal tip 24, thereby separating jaw assembly 110 from distalmost face 25 (see FIG. 6). Second actuator 156 may be operable to pivot jaw assembly 110 from the closed state (FIG. 2) towards the opened state (FIGS. 3 and 7) in response to continued distal translation relative to main body 152.

Upon positioning the target object (e.g., a tissue sample) between upper jaw 130 and lower jaw 140, first actuator 154 may be rotated to securely fasten engagement mechanism 122 with the target object. In other instances, second actuator 156 may be actuated (e.g., translated distally relative to main body 152) to extend engagement mechanism 122 distally out of jaw assembly 110 and into the target object to pierce the tissue sample prior to actuating (e.g., rotating) first actuator 154. In either instance, with the target object attached to engagement mechanism 122, second actuator 156 may be operable to pull the target object proximally into the internal cavity between upper jaw 130 and lower jaw 140 in response to a proximal translation of second actuator 156 relative to main body 152.

Second actuator 156 may be further operable to pivot jaw assembly 110 from the opened state (FIG. 3) towards the closed state (FIG. 2) in response to continued proximal translation relative to main body 152, thereby causing the plurality of teeth 132, 142 to reengage one another and sever any tissue positioned between upper jaw 130 and lower jaw 140. In this instance, the target object attached to the engagement mechanism 122 and disposed within the internal cavity of jaw assembly 110 may be separated from the surrounding tissue by the plurality of teeth 132, 142 severing and/or cutting the tissue sample from the surrounding tissue.

It should be appreciated that irrigation openings 32 may be configured to provide irrigation to distal tip 24 and into jaw assembly 110, such as towards the target object collected therein, through irrigation apertures 113 on face plate 111. Any residual material (e.g. bodily fluids and/or irrigation fluid) collected within the internal cavity of jaw assembly 110 may be released via opening 134 on upper jaw 130. Upon collecting the tissue sample within the internal cavity of jaw assembly 110, insertion portion 20 may be withdrawn from the subject with the tissue sample securely collected within jaw assembly 110 for extraction from the procedure site. Wire assembly 120 may be decoupled from handle assembly 150 and the tissue sample stored within jaw assembly 110 may be collected for analysis.

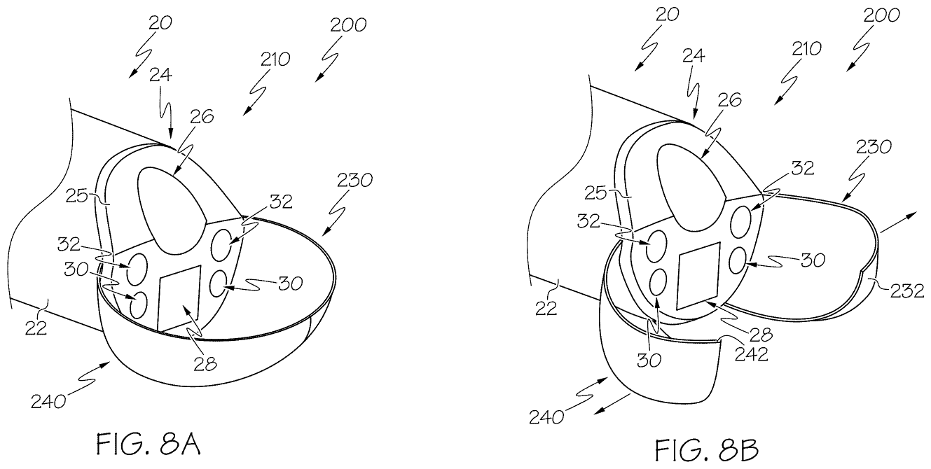

Referring now to FIGS. 8A-8B, another exemplary medical device 200 is depicted. Medical device 200 may be substantially similar to medical device 100 except for the differences explicitly described herein. Accordingly, the same reference numerals are used to identify substantially similar components. Additionally, medical device 200 may be configured and operable similar to medical device 100, such that medical device 200 may be integrated with medical system 10 in a substantially similar manner as medical device 100 shown and described above.

In the example, medical device 200 may include a jaw assembly 210, wire assembly 120 (not shown), and handle assembly 150 (see FIG. 5). Jaw assembly 210 may include a pair of jaws, such as a first (lateral) jaw 230 and a second (lateral) jaw 240. In the example, jaw assembly 210 may be sized, shaped, and/or otherwise configured as half-jaws such that the pair of jaws 230, 240 are coupled to distal tip 24 of shaft 22 only along a (half) portion of distalmost face 25. In other words, jaw assembly 210 may be disposed over distal tip 24 with the pair of jaws 230, 240 positioned over a subset (e.g., a bottom half) of distalmost face 25. In this instance, jaw assembly 210 may not fully cover distalmost face 25 such that one or more openings, channels, and/or mechanisms of distal tip 24 may be freely exposed from jaw assembly 210.

For example, referring specifically to FIG. 8A with jaw assembly 210 in a closed state, the pair of jaws 230, 240 may be engaged with one another along a bottom half of distal tip 24 such that imaging mechanism 28 and imaging mechanisms 30 may be disposed between the pair of jaws 230, 240. Working channel opening 26 and irrigation channels 32 may be uncovered by the pair of jaws 230, 240 when jaw assembly 210 is in the closed state. To facilitate navigation of insertion portion 20 towards the procedure site, jaw assembly 210 may be moved from the closed state towards an opened state to allow imaging mechanism 28 to provide visualization and lighting mechanisms 30 to provide illumination out of jaw assembly 210, as seen in FIG. 8B.

Still referring to FIG. 8B, first jaw 230 may include one or more teeth 232 and second jaw 240 may include one or more teeth 242. In the example, jaw 230 may include a single tooth 232 and second jaw 240 may include a single tooth 242. Tooth 232, 242 may define a sharpened edge and/or a blade that may be configured and operable similar to teeth 132, 142 shown and described above for severing tissue received therebetween. It should be appreciated that the half-jaw configuration of jaw assembly 210 may be configured and operable to provide an enhanced grasp of the target object (e.g., tissue sample). Additionally, jaw assembly 210 may be configured and operable to provide enhanced visualization capabilities from imaging mechanism 28 as jaw assembly 210 may be moved towards the opened state to not obstruct a field of view of imaging mechanism 28 during use.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

What is claimed is:

1. A medical device, comprising:
a wire assembly including an impediment on a distal portion of the wire assembly;
a jaw assembly coupled to the wire assembly with the impediment disposed inside the jaw assembly, wherein the jaw assembly is configured to move between a closed state and an opened state; and
a lever movably coupled to the distal portion and disposed inside the jaw assembly, wherein the lever is configured to contact an interior of the jaw assembly;
wherein the wire assembly is configured to move the jaw assembly from the closed state towards the opened state in response to the wire assembly translating distally inside the jaw assembly and moving the lever proximally until the lever engages the impediment;
wherein the lever is configured to pivot outwards relative to the jaw assembly upon engaging the impediment and push against the interior of the jaw assembly, thereby urging the jaw assembly towards the opened state.

2. The medical device of claim 1, wherein the wire assembly includes an engagement mechanism on the distal portion such that the engagement mechanism is disposed inside the jaw assembly in at least some configurations of the jaw assembly.

3. The medical device of claim 2, wherein the engagement mechanism is configured to translate distally relative to the jaw assembly from a proximal position to a distal position in response to the wire assembly translating distally.

4. The medical device of claim 3, wherein the engagement mechanism is disposed inside the jaw assembly when in the proximal position, and extends at least partially outside of the jaw assembly when in the distal position.

5. The medical device of claim 4, wherein the wire assembly is configured to rotate relative to the jaw assembly, and the engagement mechanism is configured to rotate in response to rotation of the wire assembly.

6. The medical device of claim 5, wherein the engagement mechanism is configured to pierce a tissue positioned adjacent to the jaw assembly in response to the wire assembly translating distally when the jaw assembly is in the opened state.

7. The medical device of claim 6, wherein the engagement mechanism is configured to securely engage the tissue in response to the wire assembly rotating relative to the jaw assembly.

8. The medical device of claim 7, wherein the engagement mechanism is configured to pull the tissue proximally into the jaw assembly in response to the wire assembly translating proximally relative to the jaw assembly.

9. The medical device of claim 7, wherein the jaw assembly is configured to cut the tissue engaged by the engagement mechanism and received inside the jaw assembly in response to the wire assembly translating proximally, thereby causing the jaw assembly to move from the opened state towards the closed state.

10. The medical device of claim 6, wherein the engagement mechanism includes a needle, a corkscrew, a hook, or a barbed spike.

11. The medical device of claim 1, wherein the wire assembly is configured to translate proximally relative to the jaw assembly and the lever is configured to translate distally relative to the distal portion.

12. The medical device of claim 11, wherein the impediment is configured to disengage the lever in response to the lever translating distally, such that continued translation proximal of the wire assembly is configured to pivot the lever inwards within the jaw assembly.

13. The medical device of claim 12, wherein the lever is configured to move the jaw assembly towards one another when pivoting inwards, thereby moving the jaw assembly from the opened state towards the closed state.

14. The medical device of claim 1, wherein the wire assembly extends through a shaft of an endoscope with the distal portion extending outwardly from a distal tip of the shaft, and the jaw assembly is coupled over the distal tip.

15. The medical device of claim 14, wherein the jaw assembly includes an imaging channel aligned with an imaging mechanism on the distal tip and a pair of lighting channels aligned with a pair of lighting mechanisms on the distal tip; and
wherein, with the jaw assembly coupled over the distal tip, the imaging mechanism is configured to generate images distally from the jaw assembly through the imaging channel and the pair of lighting mechanisms are configured to emit light distally from the jaw assembly through the pair of lighting channels.

16. A medical device, comprising:
a wire assembly including an impediment on a distal portion of the wire assembly;
a jaw assembly coupled to the wire assembly with the impediment disposed inside the jaw assembly, wherein the jaw assembly includes a pair of jaws configured to move between a closed state and an opened state; and
a lever movably coupled to the distal portion and disposed inside the jaw assembly, wherein the lever is configured to contact at least one of the pair of jaws;
wherein the wire assembly is configured to translate along a distal direction relative to the jaw assembly and the lever is configured to translate simultaneously towards a proximal direction relative to the distal portion; and
wherein the impediment is configured to engage the lever and inhibit further translation of the lever towards the proximal direction, such that continued translation of the wire assembly along the distal direction is configured to pivot the lever outwards from within the jaw assembly, and the lever is configured to move the at least one of the pair of jaws away from the other one of the pair of jaws when pivoting within the jaw assembly, thereby moving the pair of jaws from the closed state towards the opened state.

17. The medical device of claim 16, wherein the wire assembly includes an engagement mechanism on the distal portion that is configured to translate towards the distal direction in response to the wire assembly translating towards the distal direction, and rotate in response to the wire assembly rotating relative to the jaw assembly.

18. The medical device of claim 17, wherein the engagement mechanism is configured to pierce a tissue positioned adjacent to the jaw assembly in response to the wire assembly translating towards the distal direction when the jaw assembly is in the opened state, and securely engage the tissue in response to the wire assembly rotating relative to the jaw assembly.

19. The medical device of claim 18, wherein the engagement mechanism is configured to pull the tissue proximally into the jaw assembly in response to the wire assembly translating towards the proximal direction relative to the jaw assembly; and
wherein the jaw assembly is configured to cut the tissue engaged by the engagement mechanism in response to the wire assembly translating towards the proximal direction, thereby causing the pair of jaws to move from the opened state towards the closed state.

20. A medical system, comprising:
a shaft including a working channel and a distal tip;
a wire assembly movably disposed inside the working channel, the wire assembly including a distal portion extending outwardly from the distal tip;
a jaw assembly coupled to the wire assembly and positioned over the distal tip such that the jaw assembly abuts against a distalmost face of the distal tip, wherein the jaw assembly is configured to move between a closed state and an opened state; and
a lever movably coupled to the distal portion and disposed inside the jaw assembly and outside of the shaft, wherein the lever is configured to contact the jaw assembly;
wherein the wire assembly is configured to move the jaw assembly distally away from the distal tip in response to a distal translation relative to the shaft, and move the lever proximally within the jaw assembly in response to further distal translation relative to the shaft; and
wherein the lever is configured to pivot outwards within the jaw assembly to push against the jaw assembly, thereby moving the jaw assembly from the closed state towards the opened state.
